(19) United States
(12) Patent Application Publication
(10) Pub. No.: US 2011/0130435 A1
(43) Pub. Date: Jun. 2, 2011

(54) TIME-RELEASED MEDICATION FOR APPLYING TO CRUSTACEAN OVARIAN DEVELOPMENT

(76) Inventors: Ying-Nan CHEN, Pingtung County (TW); Kuan-Fu Liu, Pingtung County (TW)

(21) Appl. No.: 12/828,687

(22) Filed: Jul. 1, 2010

(30) Foreign Application Priority Data
Dec. 2, 2009 (TW) 098141163

(51) Int. Cl. A61K 31/4045; A61K 31/405; A61P 15/08

(52) U.S. Cl. 514/415

(57) ABSTRACT

A time-released medication for applying to crustacean ovarian development comprises a covering oil and a serotonin-related reagent. The covering oil is coated with the serotonin-related reagent and provides a sustainable releasing effect for the serotonin-related reagent. The serotonin-related reagent is selected from a group of serotonin, a precursor of serotonin, a derivative of serotonin and an agonist of serotonin. The coating relationship between the serotonin-related reagent and the covering oil makes them become a time-released medication.

### BACKGROUND OF THE INVENTION

1. Field of the Invention

The present invention relates to a time-released medication applying to Crustacean spp, particularly to a time-released medication applying to Crustaceans spp, for ovarian development.

2. Description of the Related Art

Aquaculture industry in Taiwan is well developed and famous in the world, especially in the farming of shrimps including Penaeus vannamei, Penaeus monodon, and Penaeus japonicus. According to the successful development on aquacultural diets, the farmed shrimp industry has first achieved in 1977, followed by an increasing amount of commercial production advances by years. However, due to the deterioration of cultural environment and ecology, various pathogen and infectious diseases spread out each hatchery in Taiwan leading to dramatically collapse of farming production. Recently, some healthy species of shrimps are widely imported from Thailand or South America for further selection and breeding of specific pathogen-free (SPF) larvae to rehabilitate the prosperity of shrimp industry in Taiwan. Nevertheless, most imported species are usually high in cost but still pathogen-positive which are inadequate to obtain healthy larvae for following reproductive manipulation.

Generally, the aquaculture industry of crustaceans has encountered various problems, such as the poor of the control mechanism of crustacean reproduction so that it is insufficient to maintain the reproductive performance of shrimps like farming fishes. Additionally, the aquaculture industry of shrimps in Taiwan is highly depended on wild shrimp broodstock or imported SPF larvae. The wild shrimp broodstock are usually poor in quality and susceptible to pathogen which may be worthless on reproductive manipulation. On the other hand, the imported larvae are commonly hatched in tanks for growing to maturity (about 50 to 75 gram in weight) in 10 months. However, due to the lack of natural stimulation, the shrimps hatched in tanks are poor in ovarian development and reproduction. In this situation, whatever wild shrimp broodstock or imported larvae are no longer to further apply to aquaculture industry in Taiwan.

In the conventional art, a method of unilateral eyestalk ablation tends to be the only strategy to stimulate ovarian maturation of farming shrimps. In accord with the eyestalk ablation, the secretion of vitellogenesis inhibiting hormone (VIH) from eyestalk will be limited, and accordingly the ovarian development may be initiated. On the other hand, accompanying with the eyestalk ablation, the secretion of molting inhibiting hormone (MIT) from eyestalk may also be turn down, leading to shrimps' shelling. In this situation, the shelling shrimps are less able to continually propagation without another mating. As a result, the reproductive performances of shelling shrimps are limited and valueless. It is believed that the eyestalk ablation may cause the physiological imbalances, pathogen infection and death to farming shrimps. Also, in order to promote the reproduction of the eyestalk-ablated shrimps, some fresh diets, polychaete sea worms for example, are necessary to advance the gonad development of farming shrimps which causes higher costs to farming industry. Therefore, with the conventional technique it is less efficient to control the vitellogenesis and ovarian maturation of framing shrimps.

In summary, the convention strategy of ovarian stimulation by unilateral eyestalk ablation has several weaknesses, such as high mortality, low efficiency and valueless. Most SPF broodstock have suffered from disease incidence, physiological unbalance and poor reproductive performance after unilateral eyestalk ablation. Hence it is an urgent need to develop a new strategy of ovarian stimulation to farming shrimps, for the sake of improving the imperfection of conventional technique, promoting the quality of aquaculture industry and increasing the industrial benefits of farming shrimps.

### SUMMARY OF THE INVENTION

The primary objective of this invention is to provide a time-released medication applying to Crustacean, which can avoid using eyestalk ablation as a strategy of stimulating ovarian development.

The secondary objective of this invention is to provide a time-released medication applying to Crustacean, which can avoid a high risk of lethality causing by conventional technique on initiating ovarian maturation.

Another objective of this invention is to provide a time-released medication applying to Crustacean, so that it is well applicable to stimulated ovarian development, in order to culture more economic crustaceans.

Another objective of this invention is to provide a time-released medication applying to Crustacean, so that it can be used on developing a larvae reproductive technique of crustaceans.

Another objective of this invention is to provide an injection method of the time-released medication which can be delivered into crustaceans with a more efficient and economic manner.

A time-released medication applying to Crustacean ovarian comprises a serotonin related reagent and a covering oil, for providing a sustainable releasing manner to the serotonin related reagent, wherein the serotonin related reagent is selected form a group of serotonin, a precursor of serotonin, a derivative of serotonin and a agonist of serotonin, with the coating of the covering oil resulting in a time-releasing medication.

An injection method of the time-released medication as described above comprises an anesthesia step and an implantation step, wherein the anesthesia step serving with an iced environment to crustaceans for temporarily anesthetizing and the implantation step injecting the time-released medication into a chamber space in crustaceans.

Further scope of the applicability of the present invention will become apparent from the detailed description given hereinafter. However, it should be understood that the detailed description and specific examples, while indicating preferable embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a flow chart illustrating a metabolic process of serotonin in bodies;

FIG. 2 is a diagram showing the injection method of the time-released medication on shrimp;

FIG. 3 is a line chart showing the ovarian development of female shrimps in serotonin group (C);

In the various figures of the drawings, the same numerals designate the same or similar parts.

### DETAILED DESCRIPTION OF THE INVENTION

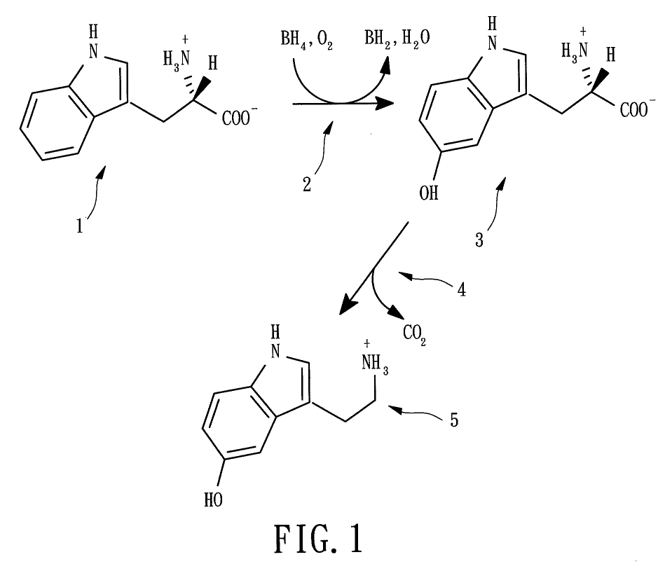

Serotonin, also called 5-Hydroxytryptamine (5-HT), is a monoamine neurotransmitter primarily found in the central nervous system (CNS) of humans and animals. Referring to the FIG. 1, illustrates that serotonin [5] is synthesized from an essential amino acid L-tryptophan [1] and its metabolite 5-hydroxytryptophan (5-HTP) [3] by a short metabolic pathway consisting of two enzymes: tryptophan hydroxylase [2] and L-amino acid decarboxylase [4] in the central nervous system of humans or animals.

Serotonin in humans and animals are mainly located in gut and CNS where it is involved in various physiological regulations including endocrine, cognitive functions and mood. It has been reported that serotonin is stimulating to vitellogenesis of Penaeus vannamei. However, serotonin taken orally does not pass into the serotonergic pathways of the central nervous system hence it is no longer to be applicable in shrimps. On the other hand, the metabolic cycle of serotonin is short and fast, and accordingly a frequent and strong dosing may need to enhance the stimulation of serotonin in bodies. In this way, frequent injecting brings about intolerable stress and pressure which may be lethal to most shrimps. Hence, it is lack of a practical implement to successfully apply serotonin on particular animals for their gonad developing.

The present invention provides a time-released medication for crustaceans, which contains a covering oil and a serotonin related reagent, wherein the covering oil is coated with the surface of the serotonin related reagent in order to present with sustainable releasing effects of the serotonin related reagent. The time-released medication is mainly applied on crustaceans, such as shrimps and crabs, for stimulating gonad development and ovarian maturation.

The covering oil mentioned in the present invention can be a glycerol, olive oil, vegetable oil or an incomplete adjuvant, which provides a coat of the serotonin related reagent providing a sustainable releasing performance.

The serotonin related reagent described above is selected from a group of a serotonin, a precursor of serotonin, a derivative of serotonin and an agonist of serotonin, preferable to the serotonin. In the present invention, the serotonin related reagent is formulated as solution in saline for further application.

For example, the time-released medication in the present invention is formulated with a proper ratio of the serotonin related reagent and the covering oil, wherein the ratio of formulation is diverse from the selection of the serotonin related reagent. For a preferable example, a time-released medication of serotonin is prepared from an emulsified mixture of a quantity of serotonin and the covering oil, in which the serotonin is caped with the covering oil resulting in several oiled sphere. As a result, the release and metabolism of serotonin are limited by the coating lay of the covering oil with a stable and long lasting manner.

The oiled sphere provides a carrier substrate for the serotonin related reagent, in order to prolong the working period and activity of the serotonin related reagent in crustaceans without continually treating. Also, with the covering of the oil sphere, the serotonin related reagent is well-delivered into particular body part in order to stimulate the gonad development of crustaceans.

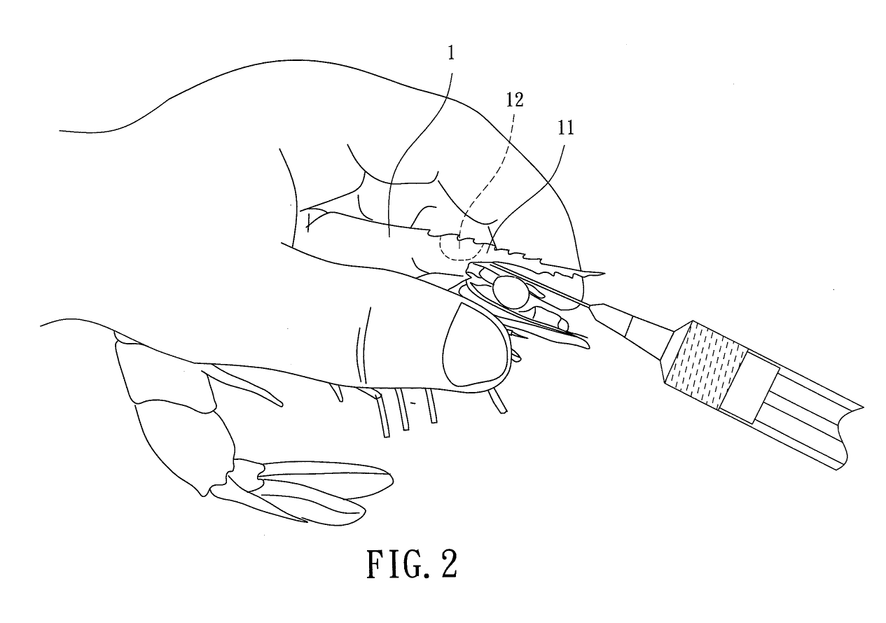

In the present invention, the time-released medication is delivered to crustaceans via an injection method described below according to the teaching of preferable embodiment. The injection method comprises an anesthesia step and implantation step. In the anesthesia step, a crustacean, a shrimp for example, is temporarily kept on an iced environment for anesthetizing to avoid the struggle of crustacean during the injection. In the implantation step, the time-released medication is prepared to inject into where it is a chamber space in the crustacean, such as a chamber localize in the base of rostrum on the head of shrimp. Referring to FIG. 2 shows a diagram of the injection method, wherein it described the time-released medication will be injected into a chamber 12 of the base of rostrum 11 on the head 1 of shrimp. In this way, the time-released medication is capable to temporally store inside the chamber space of crustaceans for persistent releasing. Also, a large amount of the time-released medication is allowed to inject into crustaceans at a time.

For further identify the effects of the time-released medication on stimulating gonad development of crustaceans, a time-released medication of serotonin [I] and a time-released medication of a precursor of serotonin -Tryptophan [II] are prepared following by the process above and injection into crustaceans for recording the ovarian maturation of crustacean.

As summarized in table 1, several female tiger shrimps, Penaeus monodon, are randomly assigned to 3 different groups including a control group (A) fed with squids and bloodworms during the studying periods, an ablation group (B) fed with squids and bloodworms after unilateral eyestalk ablation and a serotonin groups (C) fed with squids only after a treatment of the time-released medication of serotonin [I] in a dose of 1.0×10⁻⁹ to 1.0×10⁻⁵ mole for per kilogram of tiger shrimps. Also, the survival rate and ovarian development of tiger shrimps in 3 different groups in 2 weeks are reordered. The tiger shrimps, Penaeus monodon, used in the study are selected from imported larvae and free-ranged in epidemic-prevention tanks for 10 months which have SPF quality and average weight of 90.7±6 grams per shrimp.

TABLE 1
the study of the time-released medication of serotonin

| | treatments | survival | ovarian |
|---|---|---|---|
| groups | feed | rate (%) | development |
| control | non | 80 | undeveloped |
| (A) | fed with squid and bloodworm | | |
| ablation | unilateral eyestalk ablation | 60 | 33.3% stage II |
| (B) | fed with squid and bloodworm | | 33.3% stage III |
| serotonin | time-released medication of | 80 | 12.5% stage I |
| (C) | serotonin | | 12.5% stage III |
| | fed with squid | | |

In the control group (A), 80% of tiger shrimps are still lived in 2 weeks but their ovarian are all undeveloped. Compare to the control group, only 60% of tiger shrimps are kept lived after unilateral eyestalk ablation, in which 33.3% of tiger shrimps shows ovarian development in stage II and another 33.3% shows ovarian development in stage III in group (B). On the other hand, tiger shrimps in group (C) without eyestalk ablation and only fed with squid shows ovarian development in 25% shrimps (12.5% in stage I and another 12.5% in stage III of development). Moreover, around 80% tiger shrimps keep healthy after injecting of the time-released medication of serotonin in the study period. In accord with the data showed in table 1, it is suggested that the treatment of the time-released medication and eyestalk ablation are all positive to stimulate the ovarian development of tiger shrimp. However, the treatment of the time released medication shows more beneficial to the physical status of tiger shrimps, with minor mortality and better health observed on tiger shrimps of group (C).

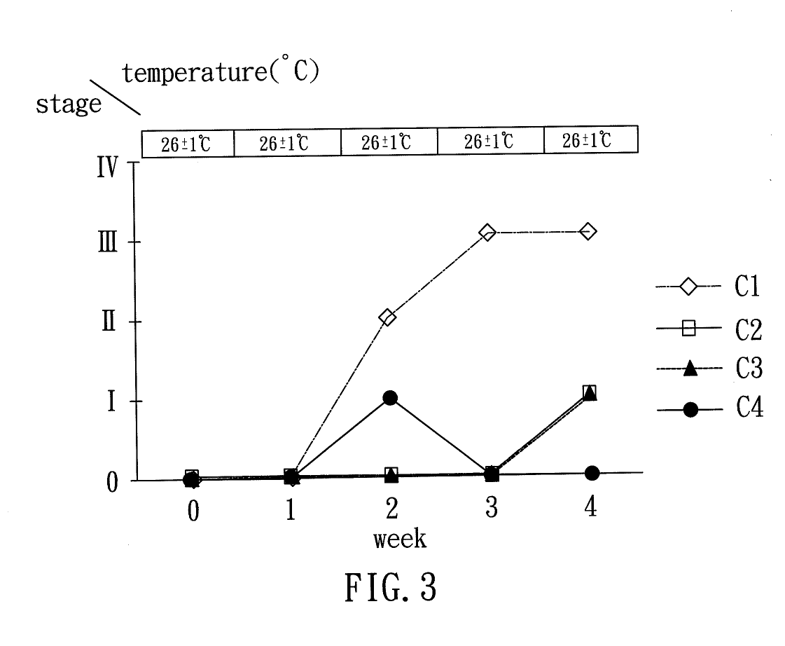

Furthermore, referring to the FIG. 3 illustrate the detail of ovarian development in group (C) shrimps, wherein, the ovarian of shrimp C1, C2, C3 and C4 have shown developing signs from the first to third week after the treatment of the time-released medication of serotonin. Besides that, the ovarian development of stage III has been shown both in the third and forth week on shrimp C1 after the treatment of the time-released medication of serotonin. It seems that the shrimp C1 has ovulated between the third and forth week and ovarian developed to stage III again. It is believed that the time-released medication in the present invention has time-releasing manner and long lasting effects, and accordingly it is sufficient to creative multipliable stimulation to gonad development of crustaceans, also to prevent the stress or pressure caused by continually injection. Hence, it seems the time-released medication in the present invention is more efficient to apply to aquaculture industry, which not only significantly advance the gonad maturation but also maintain the physical health of farming crustaceans.

Additionally, in table 2, several female tiger shrimps are randomly assigned to another 3 different groups including a control group (a) fed with squids and bloodworms during the studying periods, an ablation group (b) fed with squids and bloodworms after unilateral eyestalk ablation and a serotonin groups (c) fed with squids only after a treatment of the time-released medication of tryptophan [II] in a the same dose of 1.0×10⁻⁹ to 1.0×10⁻⁵ mole for per kilogram of tiger shrimps as the time-released medication of serotonin. Also, the survival rate and ovarian development of tiger shrimps in 3 different groups in 2 weeks are reordered.

TABLE 2
the study of the time-released medication of tryptophan

| | treatments | survival | ovarian |
|---|---|---|---|
| groups | feed | rate (%) | development |
| control | non | 80 | undeveloped |
| (a) | fed with squid and bloodworm | | |
| ablation | unilateral eyestalk ablation | 60 | 33.3% stage II |
| (b) | fed with squid and bloodworm | | 33.3% stage III |
| serotonin | time-released medication of | 100 | 10% stage I |
| (c) | tryptophan | | |
| | fed with squid | | |

In the control group (a), 80% of tiger shrimps are still lived in 2 weeks but their ovarian are all undeveloped. Compare to the control group, only 60% of tiger shrimps keep lived after unilateral eyestalk ablation, in which 33.3% of tiger shrimps shows ovarian development in stage II and another 33.3% shows ovarian development in stage III in group (b). On the other hand, tiger shrimps in group (c) without eyestalk ablation and only fed with squid shows ovarian development of stage I in 10% shrimps. Moreover, no tiger shrimps in group (c) show any physical failure after injecting of the time-released medication of tryptophan during the study period. In accord with the data showed in table 2, it is suggested that the serotonin related reagent contained in the serotonin related medication can be a precursor of serotonin, such as tryptophan, which also shows essential stimulation to ovarian development of crustaceans. Moreover, it is no longer to cause any negative physical effect on crustaceans after injection. On the other hand, tryptophan is an essential amino acid which must be taken orally from dairy diets, for that reason a delivering pathway other than injection might be applicable to the serotonin related medication of tryptophan on crustaceans as treatment.

In summarized, the time-released medication in the present invention contains a serotonin related reagent which is partially or completely suppressant to the secretion of VIH, in accord with the gonad maturation of crustaceans. Hence, based on the study of phamacodynamics, the serotonin related reagent in the present invention is serotonin, a precursor of the serotonin (ex. tryptophan), a derivative of the serotonin, an agonists of the serotonin and a partial agonist of the serotonin, which share similar enzymatic activity, suppression of VIH, also the stimulation to ovarian development of crustaceans. The time-released medication in the present invention provides a kind of oiled sphere with the serotonin related reagent coat of a covering oil oiled spheres, to perform a sustainable released manner on the serotonin related reagent. With the treatment of the time-released medication, crustaceans show significant performance on gonad development and ovarian maturation. It is sufficient to avoid the high mortality, diseases and shelling problem caused by eyestalk ablation, and then bring about more valuable effects on multiple egg production. Furthermore, it is essential for the time-release medication to apply to the aquaculture industry in order to establish a standard cultural system of crustaceans, to commercialize the production of SPF larvae of shrimps and crabs, to promote the quality of aquaculture and finally to increase the economic production of farming industry.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A time-released medication applying to Crustacean ovarian, comprising:
a serotonin related reagent; and
a covering oil for coating the serotonin related reagent in order to provide a sustained releasing effect for the serotonin related reagent;
wherein the serotonin related reagent is selected form a group of serotonin, a precursor of serotonin, a derivative of serotonin and a agonist of serotonin, with the coating of the covering oil resulting in a time-releasing medication.

2. The time-released medication applying to Crustacean ovarian as defined in claim 1, wherein the dosage of the time-related medication is 1.0×10⁻⁹ to 1.0×10⁻⁵ mole per kilogram of body weight.

3. The time-released medication applying to Crustacean ovarian as defined in claim 1, wherein the precursor of serotonin is Tryptophan;

4. The time-released medication applying to Crustacean ovarian as defined in claim 1, wherein the derivative of serotonin is 5-Hydrox-L-tryptophan.

5. The time-released medication applying to Crustacean ovarian as defined in claim 1, wherein the covering oil is selected from a group of glycerol, olive oil, vegetable oil and incomplete adjuvant.

6. An injection method of the time-released medication as defined in claim 1, comprising:
an anesthesia step, serving with an iced environment to crustaceans for temporarily anesthetizing; and
an implantation step, injecting the time-released medication as defined in claim 1 into a chamber space in crustaceans.

7. The injection method of the time-released medication as defined in claim 6, wherein injecting volume of the time-released medication in the implantation step is 0.1 to 0.5 ml.

8. The injection method of the time-released medication as defined in claim 6, wherein the crustaceans can be shrimps and crabs.

9. The injection method of the time-released medication as defined in claim 8, wherein the chamber space is localized in the base of rostrum on the head of shrimp.
